(19) United States
(12) Patent Application Publication
(10) Pub. No.: US 2024/0363207 A1
(43) Pub. Date: Oct. 31, 2024

(54) Automated System for Scanned Medical Documents Segmentation, Classification, and Matching Against Electronic Records

(71) Applicant: Google LLC, Mountain View, CA (US)

(72) Inventors: Itay Laish, Timrat (IL); Natan Potikha, Tel Aviv (IL); Rachana Fellinger, Los Angeles, CA (US); Thidanun Saensuksopa, San Francisco, CA (US); Eran Ofek, Rehovot (IL); Ayelet Benjamini, Berkeley, CA (US)

(21) Appl. No.: 18/307,481

(22) Filed: Apr. 26, 2023

(51) Int. Cl. G16H 10/60; G06F 16/215; G06F 16/23

(52) U.S. Cl. CPC G16H 10/60; G06F 16/215; G06F 16/2365

(57) ABSTRACT

Systems and methods for detecting duplications in electronic record systems are provided. A computing system can include one or more processors and a non-transitory computer-readable memory that stores instructions that, when executed by the one or more processors, cause the computing system to perform operations including accessing one or more scanned documents; converting each document of the one or more scanned documents into one or more text streams; determining one or more characteristics of each document of the one or more scanned documents; responsive to determining the one or more characteristics, generating respective embeddings associated with each document of the one or more scanned documents; and determining a respective similarity score for each document of the one or more scanned documents based, at least in part, on a similarity metric.

### FIELD

The present disclosure relates generally to document de-duplication. More particularly, the present disclosure relates to computer-implemented methods and systems for detecting duplications between scanned documents and records in electronic record systems.

### BACKGROUND

Standardized forms are commonly used by modern health systems to provide consistency and structure to medical records and documentation processes. For instance, modern medical systems often produce standardized forms such as, e.g., history and physical (H&P) forms, progress reports, discharge summaries, medical reconciliation forms, and surgical consent forms. However, because these standardized forms and operating procedures often vary between individual hospitals, doctor's offices, outpatient clinics, urgent care centers, etc., a large portion of medical communication between modern health systems and/or patients is based on a combination of printed documents, handwritten documents, faxed documents, and/or electronic documents. As such, patient health records often contain duplicate versions of the same documents (e.g., scanned versions and electronic versions).

For example, a patient visiting a first medical center (e.g., a radiology center) will obtain a printed copy of a diagnostic report that is stored electronically by the first medical center. Oftentimes, the patient will take that printed diagnostic report to a second medical center (e.g., a primary care physician's office), which creates an additional electronic version by scanning the printed copy of the diagnostic report for entry into an electronic health record (EHR) of the patient. In some instances, the second medical center will copy (e.g., type, handwrite) portions of the printed copy of the diagnostic report to create an independent electronic document, which is subsequently entered into the EHR of the patient. This can result in duplications of portions of the diagnostic report (and/or the diagnostic report as a whole) being entered into the EHR of the patient, thereby causing the EHR of the patient to include duplicate documents which require redundant use of storage.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method for detecting duplications in electronic record systems including a plurality of electronic records. The method can include obtaining, at a computing system including one or more processors, one or more scanned documents. The method can include converting, via the computing system, each document of the one or more scanned documents into one or more text streams. The method can include determining, via the computing system, one or more characteristics of each document of the one or more scanned documents. The method can include, for each document of the one or more scanned documents, generating, via the computing system, an embedding associated with the document in response to determining the one or more characteristics of each document of the one or more scanned documents. The method can include, for each document of the one or more scanned documents, determining, via the computing system, a distance metric between the embedding associated with the document and an embedding associated with each electronic record of the plurality of electronic records.

In some implementations, converting each document of the one or more scanned documents into one or more text streams can include providing, via the computing system, each document of the one or more scanned documents to a text recognition component of the computing system.

In some implementations, determining one or more characteristics of each document of the one or more scanned documents can include providing, via the computing system, the one or more text streams to a segmenter component of the computing system, and, for each document of the one or more scanned documents, determining, via the computing system, one or more characteristics of the document based, at least in part, a text stream of the one or more text streams associated with the document. In some implementations, the segmenter component of the computing system can include a rule-based model and/or an ML transformer model, and the one or more characteristics of each document comprises a document boundary. Additionally and/or alternatively, in some implementations, the one or more characteristics of each document can include a document type of a plurality of document types.

In some implementations, the embedding associated with each document of the one or more scanned documents is generated via an encoder of the computing system. In some implementations, the encoder can be further configured to generate an embedding associated with each electronic record of the plurality of electronic records. In some implementations, the encoder can include an error-resilient rule-based model.

In some implementations, the embedding associated with each document of the one or more scanned documents can be generated via an encoder-only transformer of the computing system that can be pre-trained on a corpus comprising medical notes and artificial noise. In some implementations, the encoder-only transformer can include a Bidirectional Encoder Representations from Transformers (BERT) model.

In some implementations, determining an distance metric between the embedding associated with each document of the one or more scanned documents and the embedding associated with each electronic record of the plurality of electronic records can include determining, via the computing system, a similarity metric between the embedding associated with each document of the one or more scanned documents and the embedding associated with each electronic record of the plurality of electronic records. In some implementations, the computing system can be configured to determine the distance metric based, at least in part, on a cosine similarity metric between the embedding associated with each document of the one or more scanned documents and the embedding associated with each electronic record of the plurality of electronic records. In some implementations, the computing system can be further configured to determine the distance metric based, at least in part, on the one or characteristics of each document of the one or more scanned documents. In some implementations, the one or more characteristics can include a document type of a plurality of document types, and the method can further include determining, via the computing system, a similarity threshold for each document type of the plurality of document types.

Another example aspect of the present disclosure is directed to a computing system for detecting duplications in electronic records. The computing system can include one or more processors. The computing system can include a non-transitory computer-readable memory that stores instructions that, when executed by the one or more processors, cause the computing system to perform operations. The operations can include accessing one or more scanned documents. The operations can include converting each document of the one or more scanned documents into one or more text streams. The operations can include determining one or more characteristics of each document of the one or more scanned documents. The operations can include generating respective embeddings associated with each document of the one or more scanned documents in response to determining the one or more characteristics. The operations can include determining a respective similarity score for each document of the one or more scanned documents based, at least in part, on a similarity metric between the respective embeddings associated with each document of the one or more scanned documents and respective embeddings associated with each electronic record of the plurality of electronic records.

In some implementations, the computing system can include a segmenter configured to determine the one or more characteristics of each document of the one or more scanned documents. The computing system can further include an encoder configured to generate the respective embeddings associated with each document of the one or more scanned documents. In some implementations, the similarity metric can include a cosine similarity metric. In some implementations, the one or more characteristics of each document of the one or more scanned documents can include a document boundary and a document type of a plurality of document types.

Another example aspect of the present disclosure is directed to a non-transitory computer-readable medium storing instructions that, when executed by one or more processors of a computing system, cause the one or more processors to perform operations. The operations can include obtaining, via the computing system, one or more scanned documents. The operations can include converting, via the computing system, each document of the one or more scanned documents into one or more text streams. The operations can include determining, via the computing system, one or more characteristics of each document of the one or more scanned documents. The operations can include generating, via the computing system, respective embeddings associated with each document of the one or more scanned documents in response to determining the one or more characteristics. The operations can include determining, via the computing system, a respective similarity score for each document of the one or more scanned documents based, at least in part, on a similarity metric between the respective embeddings associated with each document of the one or more scanned documents and respective embeddings associated with each electronic record of a plurality of electronic records.

Other aspects of the present disclosure are directed to various systems, apparatuses, non-transitory computer-readable media, user interfaces, and electronic devices.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE DRAWINGS

Detailed discussion of embodiments directed to one of ordinary skill in the art are set forth in the specification, which makes reference to the appended figures, in which:

FIG. 1A depicts a block diagram of an example computing system that performs document duplication detection operations according to example embodiments of the present disclosure;

FIG. 1B depicts a block diagram of an example computing system that performs document duplication detection operations according to example embodiments of the present disclosure;

FIG. 1C depicts a block diagram of an example computing system that performs document duplication detection operations according to example embodiments of the present disclosure;

FIG. 2 depicts an example framework that performs document duplication detection operations according to example embodiments of the present disclosure; and

FIG. 3 depicts a flow diagram of an example method for detecting document duplications according to example embodiments of the present disclosure.

Repeat use of reference characters in the present specification and drawings is intended to represent the same and/or analogous features or elements of the present invention.

### DETAILED DESCRIPTION

### Overview

Generally, the present disclosure is directed to computer-implemented methods and systems for reducing document duplications in electronic health record (EHR) systems. For instance, example aspects of the present disclosure provide a machine-learned-based (ML-based) model configured to detect duplications between scanned documents and their co-existing electronic counterparts. Furthermore, example aspects of the present disclosure provide a method for optimizing coverage of the scanned documents that are matched to an electronic record stored in the EHR. By providing a robust method for detecting duplications in the contents of EHR systems, redundant and duplicative patient information can be removed (e.g., hidden) or deleted from the corresponding EHR of the patient.

In particular, example aspects of the present disclosure provide an automated framework for determining and detecting duplications between scanned documents and their co-existing electronic versions in EHR systems by employing a multi-stage approach for ML-based duplication detection for scanned resources. The multi-stage approach for ML-based duplication detection provided herein can be applied to scanned documents having an image file format (e.g., PNG, JPG, TIFF, PDF, etc.). Additionally and/or alternatively, the multi-stage approach for ML-based duplication detection provided herein can be applied to other compound document types (e.g., RTF, DOCX, HTML) where the scanned document can be a concatenation of multiple documents.

In some embodiments, the multi-stage approach for ML-based duplication detection provided herein can be a four-stage automated framework. For instance, in the first stage, the framework can be configured to use a text recognition service to translate one or more scanned documents into one or more streams of raw text (e.g., which may be represented using a series of textual tokens). As noted above, the one or more scanned documents can be given in any image format including, but not limited to, PNG, JPG, TIFF, and/or PDF.

In the second stage, the framework can be configured to input the one or more streams of raw text corresponding to the one or more scanned documents produced in the first stage into a segmenter component. The segmenter component can be configured to determine one or more characteristics of each of the one or more scanned documents based at least in part on the corresponding stream of raw text produced in the first stage. For instance, the segmenter component can be configured to determine a boundary (e.g., beginning, end) of each of the one or more scanned documents. Additionally and/or alternatively, the segmenter component can be configured to determine a document type (e.g., H&P form, progress report, discharge summary, medical reconciliation form, surgical consent form) for each of the one or more scanned documents. More particularly, the segmenter can be implemented as, e.g., a rule-based model configured to utilize textual features (e.g., headers, footers, page numbers, signatures, title) and/or a transformer model to determine the one or more characteristics of the one or more scanned documents. In some embodiments, the transformer model can be an encoder-only transformer model configured to determine the document type for every section of text in each of the one or more scanned documents. Furthermore, in instances where the document type of each of the one or more scanned documents changes, a second task managed by the transformer model can be configured to indicate an end for every section of text in each of the one or more scanned documents.

In the third stage, the framework can be configured to use an encoder to generate an embedded representation (i.e., embedding) of each of the one or more scanned documents recognized by the framework in the second stage. For instance, in some embodiments, the encoder can use an error-resilient rule-based model (e.g., bag-of-words, variants of bag-of-words, etc.) to generate the embedded representations. Additionally and/or alternatively, the framework can use an encoder-only transformer (e.g., bidirectional encoder representations from transformers (BERT)) or other form of machine-learned embedding generation model. In such embodiments, the encoder-only transformer can be pre-trained on a corpus that includes, e.g., medical notes. In some embodiments, artificial noise can be added to the corpus to reflect expected recognition errors by the framework.

In the fourth stage, the framework can be configured to determine a distance metric (e.g., similarity metric) for each embedded representation generated in the third stage. For instance, subsequent to creating an embedded representation for each record in the EHR system using the same method outlined above with respect to the third stage, the framework can be configured to compare each of the one or more scanned documents to each record in the EHR system to determine the distance metric. More particularly, the framework can be configured to measure a similarity (e.g., cosine similarity) metric between the embedded representation of each of the one or more scanned documents and the embedded representations of each record in the EHR system. In some embodiments, the framework can be configured to use additional textual features to reduce the search and increase the coverage. For instance, the framework can be configured to use a signature on a document to reduce the search by author name and/or institution. Moreover, the framework can be further configured to use dates to reduce the search by a date range. Additionally, the framework can be configured to define a distinct similarity threshold for each of the document types. In this way, the framework can be configured to overcome recognition errors based on the corresponding document type determined in the second stage.

The computer-implemented methods and systems for reducing duplicate records in electronic health record (EHR) systems according to example aspects of the present disclosure provide numerous technical effects and benefits. For instance, the systems and methods described herein may provide resulting improvements to computing technology tasked with automatically detecting data duplications within databases storing electronic records. Improvements in the speed and accuracy of determining and detecting duplications between scanned documents and their co-existing electronic versions can directly improve operational speeds for computing systems. Likewise, processing and storage requirements for computing systems can be directly reduced, ultimately resulting in more efficient resource use. In this way, valuable computing resources within a computing system that would have otherwise been needed for such tasks can be reserved for other tasks (e.g., extracting additional information from patient records, increasing storage capacity, etc.).

### Example Devices and Systems

Reference now will be made in detail to embodiments, one or more examples of which are illustrated in the drawings. Each example is provided by way of explanation of the embodiments, not limitation of the present disclosure. In fact, it will be apparent to those skilled in the art that various modifications and variations can be made to the embodiments without departing from the scope or spirit of the present disclosure. For instance, features illustrated or described as part of one embodiment can be used with another embodiment to yield a still further embodiment. Thus, it is intended that aspects of the present disclosure cover such modifications and variations.

Terms used herein are used to describe the example embodiments and are not intended to limit and/or restrict the disclosure. The singular forms “a,” “an” and “the” are intended to include the plural forms as well, unless the context clearly indicates otherwise. In this disclosure, terms such as “including”, “having”, “comprising”, and the like are used to specify features, numbers, steps, operations, elements, components, or combinations thereof, but do not preclude the presence or addition of one or more of the features, elements, steps, operations, elements, components, or combinations thereof.

It will be understood that, although the terms first, second, third, etc., may be used herein to describe various elements, the elements are not limited by these terms. Instead, these terms are used to distinguish one element from another element. For example, without departing from the scope of the disclosure, a first element may be termed as a second element, and a second element may be termed as a first element.

The term “and/or” includes a combination of a plurality of related listed items or any item of the plurality of related listed items. For example, the scope of the expression or phrase “A and/or B” includes the item “A”, the item “B”, and the combination of items “A and B”.

In addition, the scope of the expression or phrase “at least one of A or B” is intended to include all of the following: (1) at least one of A, (2) at least one of B, and (3) at least one of A and at least one of B. Likewise, the scope of the expression or phrase “at least one of A, B, or C” is intended to include all of the following: (1) at least one of A, (2) at least one of B, (3) at least one of C, (4) at least one of A and at least one of B, (5) at least one of A and at least one of C, (6) at least one of B and at least one of C, and (7) at least one of A, at least one of B, and at least one of C.

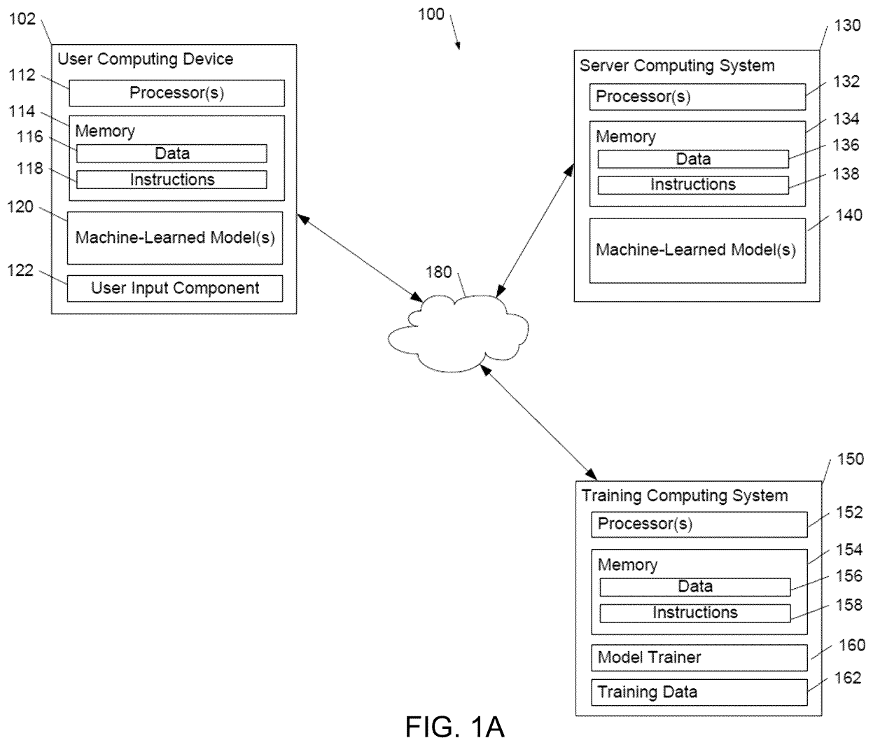

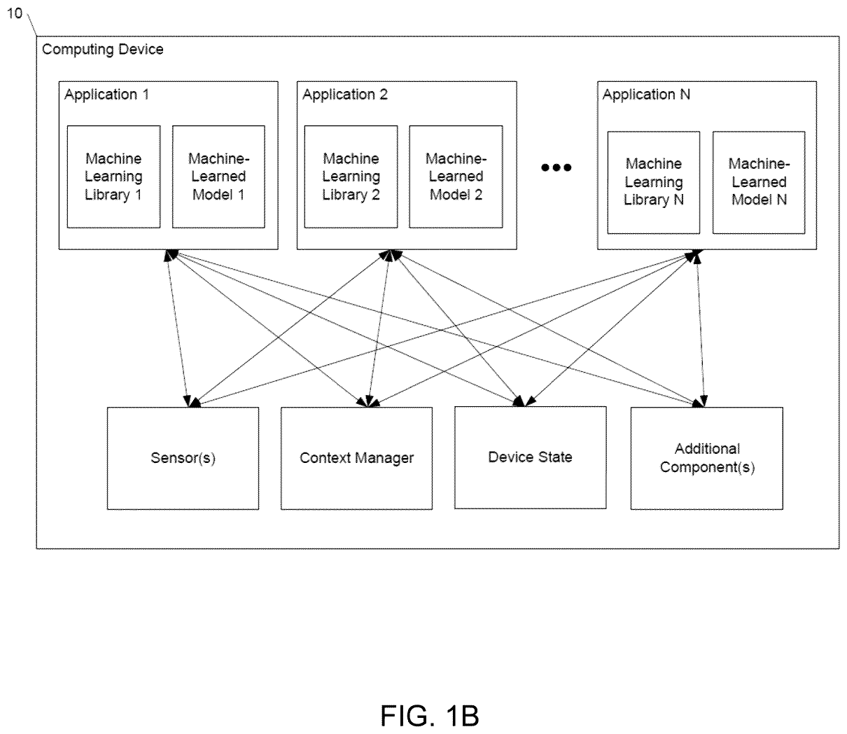

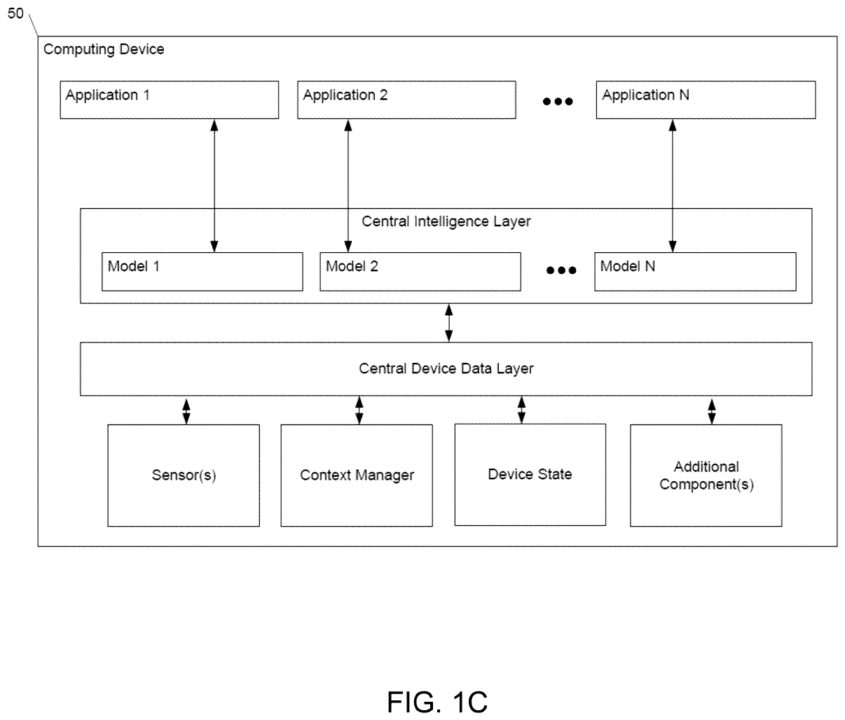

FIG. 1A depicts a block diagram of an example computing system 100 that performs document duplication detection operations according to example embodiments of the present disclosure. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, an FPGA, a controller, a microcontroller, etc.) and can be one processor or a plurality of processors that are operatively connected. The memory 114 can include one or more non-transitory computer-readable storage media, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

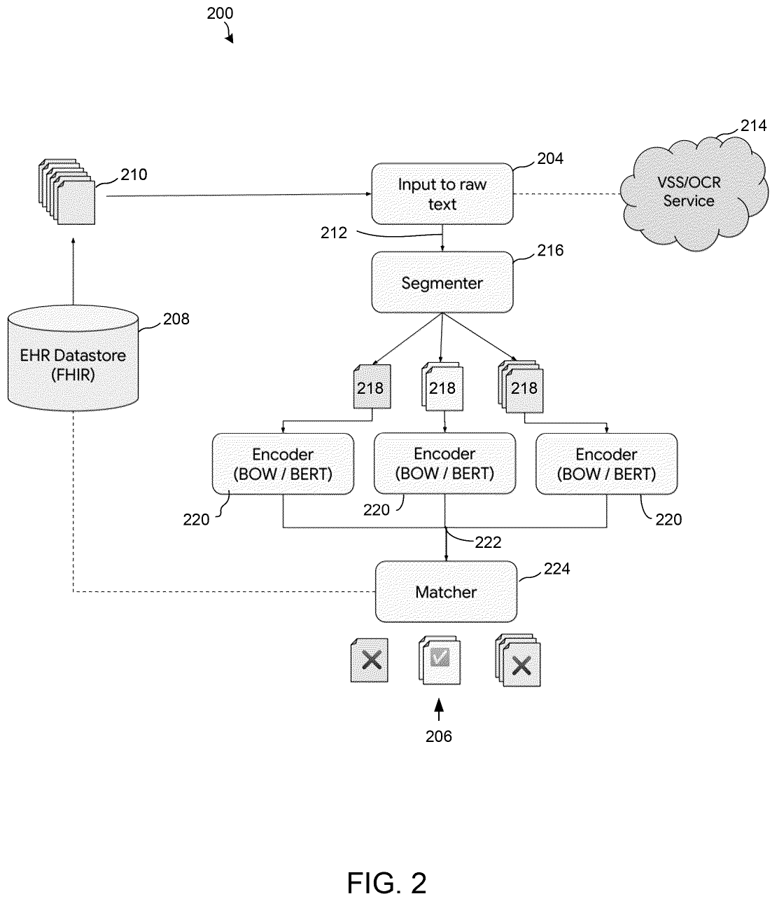

In some implementations, the user computing device 102 can store or include one or more document duplication detection models 120. For example, the document duplication detection models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Some example machine-learned models can leverage an attention mechanism such as self-attention. For example, some example machine-learned models can include multi-headed self-attention models (e.g., transformer models). Example document duplication detection models 120 are discussed with reference to FIG. 2.

In some implementations, the one or more document duplication detection models 120 can be received from the server computing system 130 over network 180, stored in the user computing device memory 114, and then used or otherwise implemented by the one or more processors 112. In some implementations, the user computing device 102 can implement multiple parallel instances of a single document duplication detection operation model 120 (e.g., to perform parallel document duplication detection operations).

More particularly, the document duplication detection model can be trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the document duplication detection model can include a matching model that is operable to determine the similarity between each of the one or more scanned documents and the plurality of electronic records stored in the electronic record system.

Additionally or alternatively, one or more document duplication detection models 140 can be included in or otherwise stored and implemented by the server computing system 130 that communicates with the user computing device 102 according to a client-server relationship. For example, the document duplication detection models 140 can be implemented by the server computing system 130 as a portion of a web service (e.g., an electronic record service). Thus, one or more models 120 can be stored and implemented at the user computing device 102 and/or one or more models 140 can be stored and implemented at the server computing system 130.

The user computing device 102 can also include one or more user input components 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, or other means by which a user can provide user input.

The server computing system 130 includes one or more processors 132 and a memory 134. The one or more processors 132 can be any suitable processing device (e.g., a processor core, a microprocessor, an ASIC, an FPGA, a controller, a microcontroller, etc.) and can be one processor or a plurality of processors that are operatively connected. The memory 134 can include one or more non-transitory computer-readable storage media, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

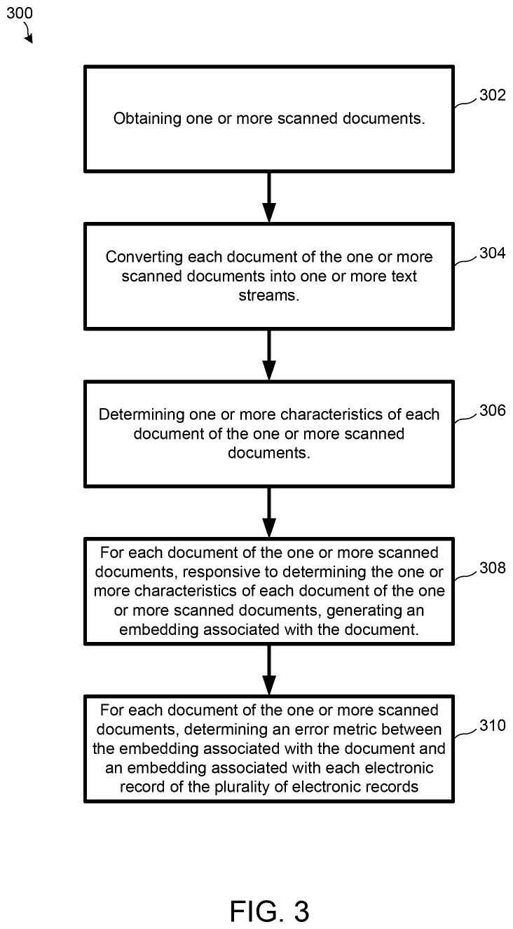

As described above, the server computing system 130 can store or otherwise include one or more document duplication detection models 140. For example, the models 140 can be or can otherwise include various machine-learned models. Example machine-learned models include neural networks or other multi-layer non-linear models. Example neural networks include feed forward neural networks, deep neural networks, recurrent neural networks, and convolutional neural networks. Some example machine-learned models can leverage an attention mechanism such as self-attention. For example, some example machine-learned models can include multi-headed self-attention models (e.g., transformer models). Example models 140 are discussed with reference to FIGS. 2-3.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, an FPGA, a controller, a microcontroller, etc.) and can be one processor or a plurality of processors that are operatively connected. The memory 154 can include one or more non-transitory computer-readable storage media, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. For example, a loss function can be backpropagated through the model(s) to update one or more parameters of the model(s) (e.g., based on a gradient of the loss function). Various loss functions can be used such as mean squared error, likelihood loss, cross entropy loss, hinge loss, and/or various other loss functions. Gradient descent techniques can be used to iteratively update the parameters over a number of training iterations.

In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the document duplication detection models 120 and/or 140 based on a set of training data 162. The training data 162 can include, for example, similar electronic records to those stored on an electronic records system. The training data 162 can further include, e.g., noise to reflect expected recognition errors by the framework.

In some implementations, if the user has provided consent, the training examples can be provided by the user computing device 102. Thus, in such implementations, the model 120 provided to the user computing device 102 can be trained by the training computing system 150 on user-specific data received from the user computing device 102. In some instances, this process can be referred to as personalizing the model.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory and executed by one or more processors. In other implementations, the model trainer 160 includes one or more sets of computer-executable instructions that are stored in a tangible computer-readable storage medium such as RAM, hard disk, or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

The machine-learned models described in this specification may be used in a variety of tasks, applications, and/or use cases.

In some implementations, the input to the machine-learned model(s) of the present disclosure can be image data. The machine-learned model(s) can process the image data to generate an output. As an example, the machine-learned model(s) can process the image data to generate an image recognition output (e.g., a recognition of the image data, a latent embedding of the image data, an encoded representation of the image data, a hash of the image data, etc.). As another example, the machine-learned model(s) can process the image data to generate an image segmentation output. As another example, the machine-learned model(s) can process the image data to generate an image classification output. As another example, the machine-learned model(s) can process the image data to generate an image data modification output (e.g., an alteration of the image data, etc.). As another example, the machine-learned model(s) can process the image data to generate an encoded image data output (e.g., an encoded and/or compressed representation of the image data, etc.). As another example, the machine-learned model(s) can process the image data to generate an upscaled image data output. As another example, the machine-learned model(s) can process the image data to generate a prediction output.

In some implementations, the input to the machine-learned model(s) of the present disclosure can be text or natural language data. The machine-learned model(s) can process the text or natural language data to generate an output. As an example, the machine-learned model(s) can process the natural language data to generate a language encoding output. As another example, the machine-learned model(s) can process the text or natural language data to generate a latent text embedding output. As another example, the machine-learned model(s) can process the text or natural language data to generate a translation output. As another example, the machine-learned model(s) can process the text or natural language data to generate a classification output. As another example, the machine-learned model(s) can process the text or natural language data to generate a textual segmentation output. As another example, the machine-learned model(s) can process the text or natural language data to generate a semantic intent output. As another example, the machine-learned model(s) can process the text or natural language data to generate an upscaled text or natural language output (e.g., text or natural language data that is higher quality than the input text or natural language, etc.). As another example, the machine-learned model(s) can process the text or natural language data to generate a prediction output.

In some implementations, the input to the machine-learned model(s) of the present disclosure can be speech data. The machine-learned model(s) can process the speech data to generate an output. As an example, the machine-learned model(s) can process the speech data to generate a speech recognition output. As another example, the machine-learned model(s) can process the speech data to generate a speech translation output. As another example, the machine-learned model(s) can process the speech data to generate a latent embedding output. As another example, the machine-learned model(s) can process the speech data to generate an encoded speech output (e.g., an encoded and/or compressed representation of the speech data, etc.). As another example, the machine-learned model(s) can process the speech data to generate an upscaled speech output (e.g., speech data that is higher quality than the input speech data, etc.). As another example, the machine-learned model(s) can process the speech data to generate a textual representation output (e.g., a textual representation of the input speech data, etc.). As another example, the machine-learned model(s) can process the speech data to generate a prediction output.

In some implementations, the input to the machine-learned model(s) of the present disclosure can be latent encoding data (e.g., a latent space representation of an input, etc.). The machine-learned model(s) can process the latent encoding data to generate an output. As an example, the machine-learned model(s) can process the latent encoding data to generate a recognition output. As another example, the machine-learned model(s) can process the latent encoding data to generate a reconstruction output. As another example, the machine-learned model(s) can process the latent encoding data to generate a search output. As another example, the machine-learned model(s) can process the latent encoding data to generate a reclustering output. As another example, the machine-learned model(s) can process the latent encoding data to generate a prediction output.

In some implementations, the input to the machine-learned model(s) of the present disclosure can be statistical data. Statistical data can be, represent, or otherwise include data computed and/or calculated from some other data source. The machine-learned model(s) can process the statistical data to generate an output. As an example, the machine-learned model(s) can process the statistical data to generate a recognition output. As another example, the machine-learned model(s) can process the statistical data to generate a prediction output. As another example, the machine-learned model(s) can process the statistical data to generate a classification output. As another example, the machine-learned model(s) can process the statistical data to generate a segmentation output. As another example, the machine-learned model(s) can process the statistical data to generate a visualization output. As another example, the machine-learned model(s) can process the statistical data to generate a diagnostic output.

In some implementations, the input to the machine-learned model(s) of the present disclosure can be sensor data. The machine-learned model(s) can process the sensor data to generate an output. As an example, the machine-learned model(s) can process the sensor data to generate a recognition output. As another example, the machine-learned model(s) can process the sensor data to generate a prediction output. As another example, the machine-learned model(s) can process the sensor data to generate a classification output. As another example, the machine-learned model(s) can process the sensor data to generate a segmentation output. As another example, the machine-learned model(s) can process the sensor data to generate a visualization output. As another example, the machine-learned model(s) can process the sensor data to generate a diagnostic output. As another example, the machine-learned model(s) can process the sensor data to generate a detection output.

In some cases, the machine-learned model(s) can be configured to perform a task that includes encoding input data for reliable and/or efficient transmission or storage (and/or corresponding decoding). For example, the task may be an audio compression task. The input may include audio data and the output may comprise compressed audio data. In another example, the input includes visual data (e.g. one or more images or videos), the output comprises compressed visual data, and the task is a visual data compression task. In another example, the task may comprise generating an embedding for input data (e.g. input audio or visual data).

In some cases, the input includes visual data and the task is a computer vision task. In some cases, the input includes pixel data for one or more images and the task is an image processing task. For example, the image processing task can be image classification, where the output is a set of scores, each score corresponding to a different object class and representing the likelihood that the one or more images depict an object belonging to the object class. The image processing task may be object detection, where the image processing output identifies one or more regions in the one or more images and, for each region, a likelihood that region depicts an object of interest. As another example, the image processing task can be image segmentation, where the image processing output defines, for each pixel in the one or more images, a respective likelihood for each category in a predetermined set of categories. For example, the set of categories can be foreground and background. As another example, the set of categories can be object classes. As another example, the image processing task can be depth estimation, where the image processing output defines, for each pixel in the one or more images, a respective depth value. As another example, the image processing task can be motion estimation, where the network input includes multiple images, and the image processing output defines, for each pixel of one of the input images, a motion of the scene depicted at the pixel between the images in the network input.

In some cases, the input includes audio data representing a spoken utterance and the task is a speech recognition task. The output may comprise a text output which is mapped to the spoken utterance. In some cases, the task comprises encrypting or decrypting input data. In some cases, the task comprises a microprocessor performance task, such as branch prediction or memory address translation.

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. In some of such implementations, the user computing device 102 can implement the model trainer 160 to personalize the models 120 based on user-specific data.

FIG. 1B depicts a block diagram of an example computing device 10 that performs according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 that performs according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model can be provided for each application and managed by the central intelligence layer. In other implementations, two or more applications can share a single machine-learned model. For example, in some implementations, the central intelligence layer can provide a single model for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Framework Arrangements

FIG. 2 depicts an example framework of an example document duplication detection model 200 according to example embodiments of the present disclosure. In some implementations, the document duplication detection model 200 is trained to receive a set of input document data 204 descriptive of one or more scanned documents and, as a result of receipt of the input document data 204, provide a distance metric 206 descriptive of a similarity between each of the one or more scanned documents and a plurality of electronic records stored in an electronic record system 208. Thus, in some implementations, the document duplication detection model 200 can include a matching model 202 that is operable to determine the similarity between each of the one or more scanned documents and the plurality of electronic records stored in the electronic record system.

More particularly, FIG. 2 depicts one or more scanned documents 210 being processed to determine whether an electronic version of each of the scanned documents 210 is stored in the electronic record system 208. For example, each of the scanned documents 210 can be translated into one or more streams of raw text 212 by text recognition block 214. In some implementations, the one or more streams of raw text can be represented using a series of textualized tokens. Furthermore, the scanned documents 210 can be provided to the model 200 in a variety of image file formats such as, e.g., PNG, JPG, TIFF, and/or PDF.

The one or more streams of raw text 212 (e.g., textualized tokens 212) can be processed by a segmenter block 216. The segmenter block 216 can be configured to process the one or more streams of raw text 212 and determine one or more characteristics of the scanned documents 210. Furthermore, the segmenter block 216 can be configured to determine one or more characteristics of each individual scanned document of the scanned documents 210.

The one or more characteristics of the scanned documents 210 can include a boundary of each of the scanned documents 210. For instance, the segmenter block 216 can determine a beginning and an end of each of the scanned documents 210. The one or more characteristics can include a classification and/or document type for each of the scanned documents 210. For instance, in implementations where the scanned documents 210 are medical documents, the segmenter block 216 can determine whether each of the scanned documents 210 is, e.g., a history and physical (H&P) form, a progress report, a discharge summary, a medical reconciliation form, a surgical consent form, and/or any other type of medical document.

In some implementations, the segmenter block 216 can include a rule-based model. The rule-based model can be configured to utilize textual features to determine the one or more characteristics. For instance, the segmenter block 216 can utilize a variety of textual features of the scanned documents 210, such as, e.g., headers, footers, page numbers, signatures, and titles in its determination of the one or more characteristics.

Additionally and/or alternatively, the segmenter block 216 can include a transformer model to determine the one or more characteristics. In some implementations, the transformer model can be an encoder-only transformer model configured to determine the document type for every section of text in each of the scanned documents 210. Furthermore, the transformer model can be configured to manage a second task to indicate an end for every section of text in each of the scanned documents 210 in instances where the document type of the scanned documents 210 changes.

Even further, in some implementations, the segmenter block 216 can include the rule-based model and the transformer model to determine the one or more characteristics of the scanned documents 210. Based at least in part on the one or more characteristics, the segmenter block 216 can generate one or more recognized streams of raw text 218. In this way, the model 200 can determine a document type for each of the scanned documents 210 subsequent to processing the one or more streams of raw text 212.

The one or more recognized streams of raw text 218 can be utilized by encoder blocks 220. Encoder blocks 220 can be configured to generate embedded representations 222 (i.e., embeddings 222) of each of the scanned documents 210 corresponding to the one or more recognized streams of raw text 218. Encoder blocks 220 can include error-resilient rule-based models configured to generate the embedded representations 222. For instance, in some implementations, encoder blocks 220 can use bag-of-words (or variants thereof) to generate the embedded representations 222.

Additionally and/or alternatively, model 200 can utilize encoder-only transformers to generate the embedded representations 222. For instance, in some implementations, the encoder-only transformers can be bidirectional encoder representations from transformers (BERT). Furthermore, model 200 can utilize other suitable forms of machine-learned embedding generation models to generate the embedded representations 222. Furthermore, the encoder blocks 220 can be pre-trained on a corpus including documents similar to those stored in the electronic record system 208. In some implementations, the corpus further includes artificial noise. The artificial noise can reflect expected recognition errors by the model 200.

The model 200 can utilize the embedded representations 222 of each of the scanned documents 210 to compare each of the scanned documents 210 to each electronic record in the electronic record system 208 in order to determine a distance metric 224 for each of the scanned documents 210. In some implementations, the model 200 can be configured to generate an embedded representation of each electronic record in the electronic record system 208 in a similar manner as set forth above with reference to the embedded representations 222 of each of the scanned documents 210. The model 200 can measure a similarity between the embedded representations 222 of each of the scanned documents 210 and the embedded representations of the electronic records of the electronic record system 208, and, as a result, the distance metrics 224 can be determined. In some implementations, the model 200 can be configured to determine a cosine similarity between the embedded representations 222 of each of the scanned documents 210 and the embedded representations of the electronic records of the electronic record system 208.

The model 200 can be configured to utilize additional textual features to reduce its search and increase coverage. For instance, in some embodiments, the model 200 can utilize a signature on a document to filter its search by, e.g., author name and/or institution. Additionally and/or alternatively, the model 200 can be configured to utilize dates on a document to filter its search by, e.g., a date range. Additionally and/or alternatively, the model 200 can be configured to define a distinct similarity threshold for each of the document types. In this way, the model 200 can be configured to dynamically overcome recognition errors based on the document type of the document.

### Example Methods

FIG. 3 depicts a flow diagram of an example method 300 to perform according to example embodiments of the present disclosure. Although FIG. 3 depicts steps performed in a particular order for purposes of illustration and discussion, the methods of the present disclosure are not limited to the particularly illustrated order or arrangement. The various steps for the method 300 can be omitted, rearranged, combined, and/or adapted in various ways without deviating from the scope of the present disclosure.

At (302), a computing system can obtain one or more scanned documents. The scanned documents can be of any image file format, such as, e.g., PNG, JPG, TIFF, PDF, etc. Additionally and/or alternatively, the scanned documents can be of any other compound document type, such as, e.g., RTF. DOCX, HTML, where the scanned document can be a concatenation of multiple documents.

At (304), the computing system can convert each document of the one or more scanned documents into one or more text streams. In some embodiments, converting each document of the one or more scanned documents into one or more text streams can include providing each document of the one or more scanned documents to a text recognition component of the computing system.

At (306), the computing system can determine one or more characteristics of each document of the one or more scanned documents. In some embodiments, to determine the one or more characteristics of each document of the one or more scanned documents, the computing system can provide the one or more text streams to a segmenter component of the computing system and, for each document of the one or more scanned documents. In some embodiments, the segmenter component of the computing system can include a rule-based model. Additionally and/or alternatively, the segmenter component can include a transformer model. In some embodiments, the one or more characteristics of each document can include a document boundary. Additionally and/or alternatively, the one or more characteristics can include a document type of a plurality of document types. Furthermore, in some embodiments, the computing system can determine the one or more characteristics of each document based, at least in part, on a text stream of the one or more text streams associated with the document.

At (308), for each document of the one or more scanned documents, the computing system can generate an embedding associated with the document in response to determining the one or more characteristics of each document of the one or more scanned documents. The embedding can be generated by an encoder of the computing system. The encoder can be further configured to generate an embedding associated with each electronic record of the plurality of electronic records. In some embodiments, the encoder can include an error-resilient rule-based model.

Additionally and/or alternatively, the embedding can be generated by an encoder-only transformer of the computing system. In some embodiments, the encoder-only transformer can be a Bidirectional Encoder Representations from Transformers (BERT) model. The encoder-only transformer can be pre-trained on a corpus including medical notes and artificial noise.

At (310), for each document of the one or more scanned documents, the computing system can determine a distance metric between the embedding associated with the document and an embedding associated with each electronic record of the plurality of electronic records. Additionally and/or alternatively, the computing system can determine a similarity metric between the embedding associated with each document of the one or more scanned documents and the embedding associated with each electronic record of the plurality of electronic records.

Additionally and/or alternatively, the computing system can determine the distance metric based, at least in part, on a cosine similarity metric between the embedding associated with each document of the one or more scanned documents and the embedding associated with each electronic record of the plurality of electronic records.

Additionally and/or alternatively, the computing system can determine the distance metric based, at least in part, on the one or more characteristics of each document of the one or more scanned documents. The one or more characteristics can include, e.g., a document type of a plurality of document types. In some implementations, the computing system can determine a similarity threshold for each document type of the plurality of document types.

### Example Operations and Uses

In some implementations, example aspects of the present disclosure provide a document duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the document duplication detection model can include a matching model that is operable to determine the similarity between each of the one or more scanned documents and the plurality of electronic records stored in the electronic record system.

Additionally and/or alternatively, example aspects of the present disclosure provide a record duplication detection model trained to receive input data descriptive of one or more digital copies and, as a result of receipt of the input data descriptive of the one or more digital copies, provide a distance metric descriptive of a similarity between each of the one or more digital copies and a plurality of digital records stored in electronic health record systems (EHR systems). Thus, in some implementations, the record duplication detection model can include a matching model that is operable to determine the similarity between each of the one or more digital copies and the plurality of digital records stored in the electronic health record systems (EHR systems).

Additionally and/or alternatively, example aspects of the present disclosure provide a report duplication detection model trained to receive input data descriptive of one or more electronic images and, as a result of receipt of the input data descriptive of the one or more electronic images, provide a distance metric descriptive of a similarity between each of the one or more electronic images and a plurality of computerized records stored in electronic medical record systems (EMR systems). Thus, in some implementations, the report duplication detection model can include a matching model that is operable to determine the similarity between each of the one or more electronic images and the plurality of computerized records stored in the electronic medical record systems (EMR systems).

Additionally and/or alternatively, example aspects of the present disclosure provide a file duplication detection model trained to receive input data descriptive of one or more scanned images and, as a result of receipt of the input data descriptive of the one or more scanned images, provide a distance metric descriptive of a similarity between each of the one or more scanned images and a plurality of e-records stored in electronic health information systems (EHIS). Thus, in some implementations, the file duplication detection model can include a matching model that is operable to determine the similarity between each of the one or more scanned images and the plurality of e-records stored in the electronic health information systems (EHIS).

Additionally and/or alternatively, example aspects of the present disclosure provide a text duplication detection model trained to receive input data descriptive of one or more digital scans and, as a result of receipt of the input data descriptive of the one or more digital scans, provide a distance metric descriptive of a similarity between each of the one or more digital scans and a plurality of electronic health records (EHRs) stored in digital health record systems. Thus, in some implementations, the text duplication detection model can include a matching model that is operable to determine the similarity between each of the one or more digital scans and the plurality of electronic health records (EHRs) stored in the digital health record systems.

Additionally and/or alternatively, example aspects of the present disclosure provide a text data duplication detection model trained to receive input data descriptive of one or more digitized documents and, as a result of receipt of the input data descriptive of the one or more digitized documents, provide a distance metric descriptive of a similarity between each of the one or more digitized documents and a plurality of electronic medical records (EMRs) stored in computerized record systems. Thus, in some implementations, the text data duplication detection model can include a matching model that is operable to determine the similarity between each of the one or more digitized documents and the plurality of electronic medical records (EMRs) stored in the computerized record systems.

Additionally and/or alternatively, example aspects of the present disclosure provide a dataset duplication detection model trained to receive input data descriptive of one or more image files and, as a result of receipt of the input data descriptive of the one or more image files, provide a distance metric descriptive of a similarity between each of the one or more image files and a plurality of electronic patient records (EPRs) stored in electronic patient record systems (EPR systems). Thus, in some implementations, the dataset duplication detection model can include a matching model that is operable to determine the similarity between each of the one or more image files and the plurality of electronic patient records (EPRs) stored in the electronic patient record systems (EPR systems).

Additionally and/or alternatively, example aspects of the present disclosure provide a record duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic health documents stored in electronic health documentation systems. Thus, in some implementations, the record duplication detection model can include a matching model that is operable to determine the similarity between each of the one or more scanned documents and the plurality of electronic health documents stored in the electronic health documentation systems.

Additionally and/or alternatively, example aspects of the present disclosure provide an entry duplication detection model trained to receive input data descriptive of one or more electronic documents and, as a result of receipt of the input data descriptive of the one or more electronic documents, provide a distance metric descriptive of a similarity between each of the one or more electronic documents and a plurality of digital health records stored in health information technology (HIT) systems. Thus, in some implementations, the entry duplication detection model can include a matching model that is operable to determine the similarity between each of the one or more electronic documents and the plurality of digital health records stored in the health information technology (HIT) systems.

Additionally and/or alternatively, example aspects of the present disclosure provide a text sequence duplication detection model trained to receive input data descriptive of one or more electronic copies and, as a result of receipt of the input data descriptive of the one or more electronic copies, provide a distance metric descriptive of a similarity between each of the one or more electronic copies and a plurality of electronic documents stored in electronic data management systems. Thus, in some implementations, the text sequence duplication detection model can include a matching model that is operable to determine the similarity between each of the one or more electronic copies and the plurality of electronic documents stored in the electronic data management systems.

Additionally and/or alternatively, example aspects of the present disclosure provide a report duplication detection model trained to receive input data descriptive of one or more digitized copies and, as a result of receipt of the input data descriptive of the one or more digitized copies, provide a distance metric descriptive of a similarity between each of the one or more digitized copies and a plurality of electronic files stored in digital health management systems. Thus, in some implementations, the report duplication detection model can include a matching model that is operable to determine the similarity between each of the one or more digitized copies and the plurality of electronic files stored in the digital health management systems.

Additionally and/or alternatively, example aspects of the present disclosure provide an input data duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the input data duplication detection model can include a matching model that is operable to determine the similarity between each of the one or more scanned documents and the plurality of electronic records stored in the electronic record system.

Additionally and/or alternatively, example aspects of the present disclosure provide a file duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the file duplication detection model can include a matching model that is operable to determine the similarity between each of the one or more scanned documents and the plurality of electronic records stored in the electronic record system.

Additionally and/or alternatively, example aspects of the present disclosure provide a text file duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the text file duplication detection model can include a matching model that is operable to determine the similarity between each of the one or more scanned documents and the plurality of electronic records stored in the electronic record system.

Additionally and/or alternatively, example aspects of the present disclosure provide a database record duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the database record duplication detection model can include a matching model that is operable to determine the similarity between each of the one or more scanned documents and the plurality of electronic records stored in the electronic record system.

Additionally and/or alternatively, example aspects of the present disclosure provide an electronic file duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the electronic file duplication detection model can include a matching model that is operable to determine the similarity between each of the one or more scanned documents and the plurality of electronic records stored in the electronic record system.

Additionally and/or alternatively, example aspects of the present disclosure provide a virtual document duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the virtual document duplication detection model can include a matching model that is operable to determine the similarity between each of the one or more scanned documents and the plurality of electronic records stored in the electronic record system.

Additionally and/or alternatively, example aspects of the present disclosure provide a data file duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the data file duplication detection model can include a matching model that is operable to determine the similarity between each of the one or more scanned documents and the plurality of electronic records stored in the electronic record system.

Additionally and/or alternatively, example aspects of the present disclosure provide a resource duplication detection model trained to receive input data descriptive of one or more scanned documents and, as a result of receipt of the input data descriptive of the one or more scanned documents, provide a distance metric descriptive of a similarity between each of the one or more scanned documents and a plurality of electronic records stored in an electronic record system. Thus, in some implementations, the resource duplication detection model can include a matching model that is operable to determine the similarity between each of the one or more scanned documents and the plurality of electronic records stored in the electronic record system.

### Additional Disclosure

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

While the present subject matter has been described in detail with respect to specific example embodiments thereof, it will be appreciated that those skilled in the art, upon attaining an understanding of the foregoing can readily produce alterations to, variations of, and equivalents to such embodiments. Accordingly, the scope of the present disclosure is by way of example rather than by way of limitation, and the subject disclosure does not preclude inclusion of such modifications, variations and/or additions to the present subject matter as would be readily apparent to one of ordinary skill in the art.

What is claimed is:

1. A computer-implemented method for detecting duplications in electronic record systems comprising a plurality of electronic records, the method comprising:
obtaining, at a computing system comprising one or more processors, one or more scanned documents;
converting, via the computing system, each document of the one or more scanned documents into one or more text streams;
determining, via the computing system, one or more characteristics of each document of the one or more scanned documents; and
for each document of the one or more scanned documents:
responsive to determining the one or more characteristics of each document of the one or more scanned documents, generating, via the computing system, an embedding associated with the document; and
determining, via the computing system, a distance metric between the embedding associated with the document and an embedding associated with each electronic record of the plurality of electronic records.

2. The computer-implemented method of claim 1, wherein converting each document of the one or more scanned documents into one or more text streams comprises providing, via the computing system, each document of the one or more scanned documents to a text recognition component of the computing system.

3. The computer-implemented method of claim 1, wherein determining one or more characteristics of each document of the one or more scanned documents comprises:
providing, via the computing system, the one or more text streams to a segmenter component of the computing system; and
for each document of the one or more scanned documents, determining, via the computing system, one or more characteristics of the document based, at least in part, a text stream of the one or more text streams associated with the document.

4. The computer-implemented method of claim 3, wherein the segmenter component of the computing system comprises a rule-based model and a transformer model.

5. The computer-implemented method of claim 4, wherein the one or more characteristics of each document comprises a document boundary.

6. The computer-implemented method of claim 4, wherein the one or more characteristics of each document comprises a document type of a plurality of document types.

7. The computer-implemented method of claim 1, wherein the embedding associated with each document of the one or more scanned documents is generated via an encoder of the computing system.

8. The computer-implemented method of claim 7, wherein the encoder is further configured to generate an embedding associated with each electronic record of the plurality of electronic records.

9. The computer-implemented method of claim 7, wherein the encoder comprises an error-resilient rule-based model.

10. The computer-implemented method of claim 1, wherein the embedding associated with each document of the one or more scanned documents is generated via an encoder-only transformer of the computing system.

11. The computer-implemented method of claim 10, wherein the encoder-only transformer is pre-trained on a corpus comprising medical notes and artificial noise.

12. The computer-implemented method of claim 11, wherein the encoder-only transformer comprises a Bidirectional Encoder Representations from Transformers (BERT) model.

13. The computer-implemented method of claim 1, wherein determining an distance metric between the embedding associated with each document of the one or more scanned documents and the embedding associated with each electronic record of the plurality of electronic records comprises determining, via the computing system, a similarity metric between the embedding associated with each document of the one or more scanned documents and the embedding associated with each electronic record of the plurality of electronic records.

14. The computer-implemented method of claim 13, wherein the computing system is configured to determine the distance metric based, at least in part, on a cosine similarity metric between the embedding associated with each document of the one or more scanned documents and the embedding associated with each electronic record of the plurality of electronic records.

15. The computer-implemented method of claim 14, wherein the computing system is further configured to determine the distance metric based, at least in part, on the one or characteristics of each document of the one or more scanned documents.

16. The computer-implemented method of claim 13, wherein the one or more characteristics comprises a document type of a plurality of document types, the method further comprising:
determining, via the computing system, a similarity threshold for each document type of the plurality of document types.

17. A computing system for detecting duplications in electronic record systems comprising a plurality of electronic records, the system comprising:
one or more processors; and
a non-transitory computer-readable memory that stores instructions that, when executed by the one or more processors, cause the computing system to perform operations, the operations comprising:
accessing one or more scanned documents;
converting each document of the one or more scanned documents into one or more text streams;
determining one or more characteristics of each document of the one or more scanned documents;
responsive to determining the one or more characteristics, generating respective embeddings associated with each document of the one or more scanned documents; and
determining a respective similarity score for each document of the one or more scanned documents based, at least in part, on a similarity metric between the respective embeddings associated with each document of the one or more scanned documents and respective embeddings associated with each electronic record of the plurality of electronic records.

18. The system of claim 17, further comprising:
a segmenter configured to determine the one or more characteristics of each document of the one or more scanned documents; and
an encoder configured to generate the respective embeddings associated with each document of the one or more scanned documents;
wherein the similarity metric comprises a cosine similarity metric.

19. The system of claim 18, wherein the one or more characteristics of each document of the one or more scanned documents comprises:
a document boundary; and
a document type of a plurality of document types.

20. A non-transitory computer-readable medium storing instructions that, when executed by one or more processors of a computing system, cause the one or more processors to perform operations, the operations comprising:
obtaining, via the computing system, one or more scanned documents;
converting, via the computing system, each document of the one or more scanned documents into one or more text streams;
determining, via the computing system, one or more characteristics of each document of the one or more scanned documents;
responsive to determining the one or more characteristics, generating, via the computing system, respective embeddings associated with each document of the one or more scanned documents; and
determining, via the computing system, a respective similarity score for each document of the one or more scanned documents based, at least in part, on a similarity metric between the respective embeddings associated with each document of the one or more scanned documents and respective embeddings associated with each electronic record of a plurality of electronic records.
